(19) United States
(12) Patent Application Publication
(10) Pub. No.: US 2010/0178262 A1
(43) Pub. Date: Jul. 15, 2010

(54) NAIL VARNISH COMPRISING A DRYING OIL, A FILM-FORMING POLYMER AND A METAL SALT

(75) Inventors: Guillaume KERGOSIEN, Chaville (FR); Henri SAMAIN, Bievres (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/634,202

(22) Filed: Dec. 9, 2009

Related U.S. Application Data
(60) Provisional application No. 61/202,023, filed on Jan. 21, 2009.
(60) Provisional application No. 61/153,319, filed on Feb. 18, 2009.

(30) Foreign Application Priority Data
Dec. 17, 2008 (FR) 08 58697
Dec. 23, 2008 (FR) 08 59035

(51) Int. Cl. A61K 8/92; A61Q 3/02

(52) U.S. Cl. 424/61

(57) ABSTRACT

A subject-matter of the present invention is a nail varnish, characterized in that it comprises at least one drying oil, at least one film-forming polymer and at least one metal salt.

Another subject-matter of the present invention is a cosmetic method for making up and/or for the nontherapeutic care of the nails comprising the application, to the nails, of at least one layer of the said nail varnish.

A subject-matter of the present invention is a nail varnish comprising at least one drying oil, at least one film-forming polymer and at least one metal salt.

This nail varnish composition can be applied to human nails or else to false nails.

The present invention additionally relates to the corresponding method for making up and/or for the nontherapeutic care of the nails.

The coloured or transparent nail varnish composition can be employed as varnish base or base coat, as product for making up the nails, as finishing composition, also known as top coat, to be applied to the product for making up the nails, or else as product for the cosmetic care of the nails.

It is commonplace to use, in nail varnish compositions, film-forming agents in order to obtain, after deposition on the nail, a resistant film exhibiting good hold. Currently, nitrocellulose still remains the commonest film-forming agent used in nail varnishes exhibiting optimized properties of hold.

However, these nail varnishes comprising nitrocellulose exhibit the disadvantage of not being stable towards heat and on storage.

This results in particular in yellowing of these compositions over time.

Furthermore, the introduction of large amounts of nitrocellulose conditions the composition of the organic solvent phase of the nail varnish, which then has to be compatible with this film-forming agent.

The present invention has specifically the aim of providing a nail varnish composition comprising a reduced content of nitrocellulose, indeed even devoid of nitrocellulose, which exhibits good stability over time and which makes possible the formation of a homogeneous, glossy and/or nonbrittle film.

The inventors have thus discovered that it is possible to obtain such a composition comprising less than 5% by weight of nitrocellulose dry matter with respect to the total weight of the composition, indeed even devoid of nitrocellulose, by using at least one drying oil, at least one film-forming polymer and at least one metal salt.

Thus, according to one of its aspects, a subject-matter of the present invention is a nail varnish comprising at least one drying oil, at least one film-forming polymer and at least one metal salt.

Such a nail varnish makes possible in particular good stability over time and makes it possible to obtain a solid, glossy and/or nonbrittle film.

According to another aspect, a further subject-matter of the invention is a cosmetic method for making up and/or for the nontherapeutic care of the nails comprising the application, to the nails, of at least one layer of a nail varnish as defined above.

According to yet another of its aspects, a further subject-matter of the invention is the use of a nail varnish as defined above in order to obtain, after deposition on the nail, a homogeneous, glossy and nonbrittle film.

The nail varnish according to the invention comprises a cosmetically acceptable medium, that is to say a nontoxic medium capable of being applied to human keratinous substances, in particular the nails.

The present invention also relates to a made-up synthetic support comprising a make-up obtained by the method of the invention.

The nail varnish according to the present invention can take different forms, either a conventional nail varnish form, that is to say that it exhibits a liquid or gel texture, or an article form which is affixed to and adjusted on the nails, as set out below.

According to one of its aspects, the nail varnish according to the invention exhibits a liquid or gel texture; it differs in particular from a soft article or patch, comprising at least one layer of polymer and a layer of adhesive, intended to be adhesively bonded to and adjusted by virtue of its deformability properties on the nail.

In other words, the nail varnish according to this aspect of the invention is typically applied in the form of superimposed layers at the surface of the nails or false nails to be made up, for example using a brush.

According to yet another of its aspects, a subject-matter of the present invention is a soft article intended to be applied to the nails and/or false nails, in order to make them up and/or to care for them, comprising at least one polymeric layer, characterized in that the said polymeric layer comprises at least one drying oil, at least one film-forming polymer and at least one metal salt.

The said soft article is advantageously introduced into a substantially airtight packaging, the packaging being such that the article is found therein stored with the exclusion of atmospheric oxygen. This soft article is called, for reasons of simplicity, in the continuation of the description, “not completely crosslinked soft article”.

According to this aspect of the invention, the not completely crosslinked soft article only acquires its definitive stiffness after application to the nail and crosslinking, by simple exposure to ambient air.

The present invention also relates to a method for making up and/or for caring for the nails and/or false nails comprising at least one stage which consists in applying, to a natural nail and/or false nail, a soft article according to the invention, whether considered in the crosslinked state or in the not completely crosslinked state.

The present invention additionally relates to a product for making up and/or caring for the nails and/or false nails comprising, in a substantially airtight packaging, at least one article in accordance with the invention, the packaging being such that the article is found therein stored in a partially dry form. It can be in the crosslinked state or in the not completely crosslinked state. This soft article is called, for reasons of simplicity, in the continuation of the description, “partially dry soft article”.

According to this aspect of the invention, the partially dry soft article only acquires a completely dry appearance, and thus its definitive shape, after application to the nail and drying, by simple exposure to ambient air.

Finally, the present invention relates to a method for making up and/or caring for the nails and/or false nails comprising at least one stage which consists in applying, to a natural nail and/or false nail, a soft article according to the invention, whether considered in the dry state or in the partially dry state.

According to one embodiment, the soft article in accordance with the present invention can be provided in various forms, such as a star, a square, a circle, and the like.

Within the meaning of the present invention, the term “partially dry” is understood to describe the fact that the article obtained after formation of the film is not completely devoid of the residual solvent. In particular, it has a dry matter content of greater than 80% by weight, more particularly of greater than 85% by weight, and of less than 95% by weight, with respect to its total weight.

In an alternative fowl, the article can comprise a superimposition of polymeric layers.

In another alternative form, the article can comprise one or more adhesive layers in addition to the polymeric layer or layers.

A soft article according to the invention is targeted at providing a form of making up and/or caring for the nails or false nails exhibiting an adhesiveness, the composition of which makes possible a satisfactory softness and a satisfactory capacity for elongation at the time of the application, and a satisfactory stiffness after drying.

In fact, it has been demonstrated that the use, in order to form a polymeric layer of a soft article for making up and/or caring for the nails, of a drying oil, of a film-forming polymer and of a metal salt, in order to form a polymeric layer of a soft article for making up and/or caring for the nails and/or false nails, makes it possible to form soft films at the time of the application which can thus be stretched easily during the application to the nails and become harder after drying for several hours.

I—Nail Varnish Composition in the Liquid or Gelled Form

Drying Oils

A nail varnish according to the invention comprises at least one drying oil.

“Drying oil” is understood to denote an oil which, when spread as a thin layer and then exposed to the air, is converted into a thin solid film.

In particular, “drying oil” is understood to denote, in the context of the present invention, oils and preferably triglycerides comprising conjugated double bonds, preferably comprising at least two conjugated double bonds and preferably comprising at least three conjugated double bonds.

The drying oils according to the invention can be of natural origin.

Advantageously, the drying oil can be chosen from drying vegetable oils, such as linseed oil, China wood oil (or tung oil), oiticica oil, vernonia oil, poppy seed oil, pomegranate seed oil or calendula oil; the esters of these vegetable oils; the alkyd resins obtained from these vegetable oils; and their mixtures.

Alkyd resins are polyesters comprising hydrocarbon chains of fatty acids, obtained in particular by polymerization of polyols and polyacids or their corresponding anhydrides in the presence of fatty acids. These fatty acids are present, in particular in the form of triglycerides, in the majority of natural oils, such as in particular the abovementioned oils.

The drying oil suitable for the implementation of the present invention can be modified by chemical reaction.

In particular, it can be refined and/or partially polymerized. Mention may be made, as such, of blown oils and stand oils, maleinized oils, epoxidized oils or boiled oils.

According to a specific embodiment of the invention, the siccative oil of the invention is different from epoxidized oils.

According to a specific embodiment of the invention, the drying oil is a refined linseed oil.

An oil can be refined in particular in three successive stages.

The refined linseed oil according to the invention can thus result from a degumming stage, in order to obtain in particular a demucilaginated oil, followed by a decolouration stage, in particular in order to whiten it, and then by a neutralization stage.

According to a specific embodiment of the invention, the drying oil of the invention is a linseed oil modified according to at least one of the three above-mentioned stages, in other words which has been subjected either to a degumming stage, or to a decolouration stage, or to a neutralization stage, or to a succession of these stages.

According to yet another specific form of the invention, the drying oil of the invention is a modified linseed oil which has been subjected to two stages among those considered above.

According to another specific embodiment of the invention, the drying oil is a blown linseed oil.

The blowing of an oil is characterized in particular by a polymerization of the said oil with atmospheric oxygen. The blown oil can in particular be obtained by blowing air through the heated oil.

According to a specific embodiment, the cosmetic compositions according to the invention comprise from 5 to 95% by weight, in particular from 5 to 40% by weight, especially from 5 to 15% by weight, of drying oil, with respect to the total weight of the composition.

Metal Salts

A nail varnish in accordance with the invention comprises at least one metal salt and preferably at least two separate metal salts.

The combination of at least one drying oil with one (or more) metal salt(s) makes it possible in particular, during drying, to form a film by crosslinking with atmospheric oxygen.

The metal salt(s) in particular accelerate(s) the drying of natural drying oils.

Advantageously, the metal salt used comprises less than 5% of cobalt, with respect to the total weight of the metal in the metal salts, indeed even is advantageously devoid of cobalt, in particular in order to obtain a more favourable toxicological profile.

According to a specific embodiment, the metal salt(s) in accordance with the invention is (are) chosen from manganese, calcium, zirconium, zinc, strontium, lithium, cerium and vanadium salts.

According to a specific embodiment, the metal salt is an organic salt. It can in particular exhibit the following formula (I):

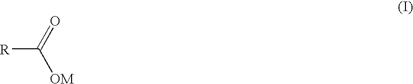

in which:

M is chosen from manganese, calcium, zirconium, zinc, strontium, lithium, cerium and vanadium; and

R is a linear or branched (C₁-C₂₅)alkyl radical which can optionally comprise 1 to 3 unsaturations.

According to a specific form, R is a linear or branched (C₆-C₂₀)alkyl radical which can optionally comprise one or two unsaturations.

According to a specific form, the metal salt can take the octoate, linoleate, octanoate, oleate, stearate, laurate or naphthenate foam

According to a specific embodiment, a nail varnish in accordance with the invention comprises at least one manganese salt.

According to another specific embodiment of the invention, the nail varnish composition according to the invention comprises several metal salts, in particular with the aim of increasing the rate of crosslinking of the drying oil.

According to a specific embodiment of the invention, the nail varnish cosmetic compositions according to the invention comprise from 0.001 to 2% by weight, in particular from 0.01 to 1% by weight, especially from 0.1 to 0.5% by weight, of metal, with respect to the solids content.

Examples of commercial metal salt products are given below: Octa-Soligen Manganese 6HS, Octa-Soligen Zirconium 12 HS and Octa-Soligen Calcium 5 HS, sold by OMG Borchers GmbH.

The indication xHS (6HS, 12HS and 5HS) employed above indicates that the commercial product or starting material comprises x % of metal, with respect to the total weight of the commercial product or metal salt.

According to a specific form of the invention, a nail varnish according to the invention can comprise at least two different metal salts chosen from the salts as defined above.

According to another specific form of the invention, the metal salts of a nail varnish of the invention can comprise at least one primary metal salt and at least one secondary metal salt, indeed even can consist of a primary metal salt and a secondary metal salt.

In the context of this specific embodiment,
the primary metal salt can be chosen from manganese salts, cerium salts and vanadium salts and more particularly is a manganese salt; and
the secondary metal salt can be chosen from zirconium, zinc, lithium and strontium salts and is more particularly a zirconium salt.

In the context of this specific embodiment, the metal salts of a nail varnish of the invention can comprise at least one manganese salt and at least one zirconium salt.

According to yet another preferred embodiment of the invention, a nail varnish of the invention can comprise at least one primary metal salt as defined above, at least one secondary metal salt as defined above and at least one auxiliary metal salt.

In the context of the last specific embodiment, the auxiliary metal salt can be chosen from calcium salts.

According to a particularly preferred embodiment, the metal salts of a nail varnish of the invention can comprise at least one manganese salt, at least one zirconium salt and at least one calcium salt, indeed even can consist of a manganese salt, a zirconium salt and a calcium salt.

In the context of this particularly preferred embodiment, the manganese salt can in particular promote the surface drying and the complete drying of the film; the zirconium salt can promote the in-depth drying, bringing about uniform drying of the film; and the calcium salt can make it possible to develop a synergistic effect between the manganese salt and the zirconium salt.

Still according to this particularly preferred embodiment, the nail varnish composition of the invention can comprise at least one primary metal salt as defined above in a content ranging from 0.005 to 0.1% by weight of metal, with respect to the solids content, in particular from 0.01 to 0.05% by weight of metal, with respect to the solids content. It can in addition comprise at least one secondary metal salt as defined above in a content ranging from 0.005 to 0.8% by weight of metal, with respect to the solids content, in particular from 0.03 to 0.5% by weight of metal, with respect to the solids content. Finally, it can additionally comprise at least one auxiliary metal salt as defined above in a content ranging from 0.03 to 0.3% by weight of metal, with respect to the solids content.

Characterization of the Solids Content

The dry matter content, commonly known as “solids content”, that is to say the content of non-volatile material, can be measured in various ways. Mention may be made, for example, of the methods by drying in an oven or the methods by drying by exposure to infrared radiation.

Preferably, the amount of dry matter of the nail varnish compositions according to the invention is measured by heating the sample with infrared rays with a wavelength of 2 μm to 3.5 μm. The substances present in the said compositions which have a high vapour pressure evaporate under the effect of this radiation. The measurement of the loss in weight of the sample makes it possible to determine “the solids content” of the composition. These measures are carried out using a commercial infrared dryer LP16 from Mettler. This technique is fully described in the documentation of the device supplied by Mettler.

The measurement protocol is as follows:

Approximately 1 g of the composition is spread over a metal dish. The latter, after introducing into the dryer, is subjected to a set temperature of 120° C. for one hour. The wet weight of the sample, corresponding to the initial weight, and the dry weight of the sample, corresponding to the weight after exposure to the radiation, are measured using a precision balance.

The dry matter content is calculated in the following way:

Solids content=100×(dry weight/wet weight).

The nail varnish compositions according to the invention are characterized by a dry matter content of between 10 and 60% by weight and more particularly between 20 and 40% by weight.

Film-Forming Polymer

A nail varnish according to the invention comprises at least one film-forming polymer.

“Film-forming polymer” denotes, within the meaning of the present invention, a polymer capable of forming, by itself alone or in the presence of an additional agent which is able to form a film, an isolable film, in particular a continuous and adherent film, on a support, in particular on the nails.

In particular, the film-forming polymer of the invention is different from the siccative oils as above-described.

Use may be made, in the composition, of just one film-forming polymer or of a blend of film-forming polymers.

This film-forming polymer can be chosen from the group consisting of synthetic polymers of radical type or of polycondensate type, polymers of natural origin and their blends.

A film-forming polymer suitable for the invention can be chosen in particular from:
polysaccharide derivatives, such as cellulose derivatives or guar gum derivatives. A polysaccharide derivative suitable for the invention can be a polysaccharide ester or alkyl ether.

“Polysaccharide ester or alkyl ether” denotes a polysaccharide formed of repeat units comprising at least two identical or different rings and exhibiting a degree of substitution per saccharide unit of between 1.9 and 3, preferably of between 2.2 and 2.9 and more particularly of between 2.4 and 2.8. Substitution denotes the functionalization of the hydroxyl groups to give ester and/or alkyl ether functional groups and/or the functionalization of the carboxyl groups to give ester functional groups.

In other words, it can be a polysaccharide partially or completely substituted by ester and/or alkyl ether groups. Preferably, the hydroxyl groups can be substituted by ester functional groups and/or alkyl ether functional groups comprising from two to four carbon atoms.

Mention may in particular be made of cellulose esters, such as cellulose acetate, cellulose acetate butyrates or cellulose acetate propionates; or cellulose alkyl ethers, such as ethyl celluloses and ethyl guars;
synthetic polymers, such as polyurethanes, acrylic polymers, vinyl polymers, polyvinyl butyrals, alkyd resins and ketone/aldehyde resins, resins resulting from aldehyde condensation products, such as arylsulphonamide/formaldehyde resins, for example toluenesulphonamide/formaldehyde resin, arylsulphonamide/epoxy resins or ethyl tosylamide resins;
polymers of natural origin, such as plant resins, such as dammars, elemi, copals or benzoin; or gums, such as shellac, sandarac gum and mastic gum.

Use may in particular be made, as film-forming polymer, of the toluenesulphonamide/formaldehyde resins “Ketjentflex MS80” from Akzo or “Santolite MHP” or “Santolite MS 80” from Faconnier or “Resimpol 80” from Pan Americana, the alkyd resin “Beckosol ODE 230-70-E” from Dainippon, the acrylic resin “Acryloid B66” from Röhm & Haas, the polyurethane resin “Trixene PR 4127” from Baxenden or the acetophenone/formaldehyde resin sold under the reference Synthetic Resin SK by Degussa.

According to a specific embodiment, the film-forming polymer is chosen from polysaccharides and polysaccharide derivatives, preferably from polysaccharide ethers and esters, in particular comprising from two to four carbon atoms, and is more preferably chosen from cellulose acetate butyrates, cellulose acetate propionates, cellulose acetate, ethyl celluloses, ethyl guars and their blends.

According to a particularly preferred embodiment, the film-forming polymer is chosen from ethyl guar and ethyl cellulose.

According to a specific embodiment, a nail varnish of the invention comprises a total content of film-forming polymer of between 1 and 60% by weight, in particular between 2 and 30% by weight, especially between 5 and 15% by weight, with respect to the total weight of the nail varnish.

According to a preferred embodiment, a nail varnish composition according to the invention comprises less than 5% by weight of nitrocellulose dry matter, with respect to the total weight of the composition, preferably less than 3% by weight, preferably less than 1.5% by weight and preferably less than 1% by weight.

According to a particularly preferred embodiment, the nail varnish composition of the invention is completely devoid of nitrocellulose.

According to a specific form of the invention, a nail varnish according to the invention comprises at least ethyl guar, at least blown linseed oil and at least manganese, zirconium and calcium salts.

Physiologically Acceptable Medium

The composition of the invention additionally comprises a physiologically acceptable medium. This term denotes a nontoxic medium capable in particular of being applied to the nails.

This medium can be of organic solvent type or of dispersion of an organic phase in an aqueous continuous phase type.

The physiologically acceptable medium of the composition generally comprises at least one volatile solvent. This volatile solvent can be chosen in particular from volatile organic solvents, water and their mixtures.

Organic Solvent Medium

The nail varnish composition according to the invention can comprise at least one organic solvent medium comprising one or more nonaqueous compounds which are liquid at ambient temperature, also known as oils or organic solvents.

The organic solvent can be chosen from:
ketones which are liquid at ambient temperature, such as methyl ethyl ketone, methyl isobutyl ketone, diisobutyl ketone, isophorone, cyclohexanone or acetone; alcohols which are liquid at ambient temperature, such as ethanol, isopropanol, diacetone alcohol, 2-butoxyethanol or cyclohexanol;
propylene glycol ethers which are liquid at ambient temperature, such as propylene glycol monomethyl ether, propylene glycol monomethyl ether acetate or dipropylene glycol mono(n-butyl)ether;
cyclic ethers, such as γ-butyrolactone;
short chain esters (having from 3 to 8 carbon atoms in total), such as ethyl acetate, methyl acetate, propyl acetate, isopropyl acetate, n-butyl acetate, isopentyl acetate, methoxypropyl acetate, t-butyl acetate or butyl lactate;
ethers which are liquid at ambient temperature, such as diethyl ether, dimethyl ether or dichlorodiethyl ether;
alkanes which are liquid at ambient temperature, such as decane, heptane, dodecane or cyclohexane;
alkyl sulphoxides, such as dimethyl sulphoxide;
aldehydes which are liquid at ambient temperature, such as benzaldehyde or acetaldehyde;
ethyl 3-ethoxypropionate;
carbonates, such as propylene carbonate or dimethyl carbonate;
acetals, such as methylal;
and their mixtures.

The organic medium can comprise at least one volatile silicone oil.

“Volatile oil” is understood to mean, within the meaning of the invention, an oil capable of evaporating on contact with keratinous substances in less than one hour at ambient temperature and atmospheric pressure. The volatile organic solvent or solvents and the volatile oils of the invention are volatile cosmetic organic solvents and oils which are liquid at ambient temperature and which have a nonzero vapour pressure, at ambient temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The volatile silicone oils can be linear or cyclic volatile silicone oils, in particular those having a viscosity ≧8 centistokes (8×10⁻⁶ m²/s) and having in particular from 2 to 7 silicon atoms, these silicones optionally comprising alkyl or alkoxy groups having from 1 to 10 carbon atoms. Mention may in particular be made, as volatile silicone oils which can be used in the invention, of octamethylcyclotetrasiloxane, deca-methylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Mention may also be made of the volatile linear alkyltrisiloxane oils of general formula (I):

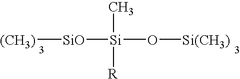

where R represents an alkyl group comprising from 2 to 4 carbon atoms, one or more hydrogen atoms of which can be replaced by a fluorine or chlorine atom.

Mention may be made, among the oils of general formula (I), of:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is respectively a butyl group, a propyl group or an ethyl group.

Preferably, the solvent is chosen from short chain esters having from 3 to 8 carbon atoms in total, such as ethyl acetate, methyl acetate, propyl acetate, isopropyl acetate, n-butyl acetate, isopentyl acetate, methoxypropyl acetate, butyl lactate and their mixtures.

The organic solvent medium can preferably be ethyl acetate or ethanol.

The organic solvent medium can represent from 10 to 95% by weight, preferably from 15 to 80% by weight and better still from 20 to 70% by weight, with respect to the total weight of the composition.

Aqueous Medium

The composition according to the invention can also comprise at least one aqueous medium, constituting an aqueous phase, which can form a continuous phase in which is dispersed an organic phase, such as described above.

The aqueous phase can be composed essentially of water; it can also comprise a mixture of water and of water-miscible organic solvent(s) (miscibility in water of greater than 50% by weight at 25° C.), such as lower monoalcohols having from 1 to 5 carbon atoms, for example ethanol or isopropanol, glycols having from 2 to 8 carbon atoms, such as propylene glycol, ethylene glycol, 1,3-butylene glycol or dipropylene glycol, C₃-C₄ ketones or C₂-C₄ aldehydes.

The aqueous continuous phase (water and optionally water-miscible organic solvent) can be present in a proportion varying from 30 to 90% by weight, preferably from 50 to 80% by weight, with respect to the total weight of the composition.

According to another embodiment, the composition comprises a continuous organic solvent medium and less than 10% by weight of water, preferably less than 5% by weight of water, with respect to the total weight of the composition.

Additional Agent which is Able to Form a Film

An additional agent which is able to form a film may be provided in order to improve the film-forming properties of the nail varnish composition.

Such an additional agent which is able to form a film can be chosen from any compound known to a person skilled in the art as being capable of fulfilling the desired purpose and can in particular be chosen from plasticizing agents and coalescence agents for the film-forming polymer or polymers.

Thus, the composition can additionally comprise at least one plasticizing agent and/or one coalescence agent. Mention may in particular be made, alone or as a mixture, of the usual plasticizers and coalescence agents, such as:
glycols and their derivatives, such as diethylene glycol ethyl ether, diethylene glycol methyl ether, diethylene glycol butyl ether or diethylene glycol hexyl ether, ethylene glycol ethyl ether, ethylene glycol butyl ether or ethylene glycol hexyl ether;
glycol esters;
propylene glycol derivatives and in particular propylene glycol phenyl ether, propylene glycol diacetate, dipropylene glycol ethyl ether, tripropylene glycol methyl ether and diethylene glycol methyl ether, or propylene glycol butyl ether;
acid esters, in particular carboxylic acid esters, such as citrates, phthalates, adipates, carbonates, tartrates, phosphates or sebacates;
oxyethylenated derivatives, such as oxyethylenated oils, in particular vegetable oils, such as castor oil;
oils of natural origin, in particular non-drying oils, chosen from oils comprising at least one fatty acid chosen from caprylic acid, capric acid, lauric acid, myristic acid, palmitic acid, stearic acid, oleic acid, ricinoleic acid, linoleic acid, linolenic acid, arachidic acid, gadoleic acid, behenic acid, erucic acid, brassidic acid, cetoleic acid, lignoceric acid or nervonic acid. In particular, these oils are chosen from triglycerides composed of esters of fatty acids and of glycerol, the fatty acids of which can have varied chain lengths from C₄ to C₂₄, it being possible for the latter to be linear or branched and saturated or unsaturated. These oils are in particular heptanoic or octanoic triglycerides, groundnut, babassu, coconut, grape seed, cottonseed, maize, maize germ, mustard seed, palm, rapeseed, sesame, soybean, sunflower, wheat germ, canola, apricot, mango, castor, shea, avocado, olive, sweet almond, almond, peach, walnut, hazelnut, macadamia, jojoba, alfalfa, poppy, pumpkinseed, cucumber, blackcurrant, evening primrose, millet, barley, guinea, rye, safflower, candlenut, passionflower, musk rose or shea butter oils or triglycerides of caprylic/capric acids, and
their mixtures.

The type and the amount of plasticizing agent and/or coalescence agent can be chosen by a person skilled in the art on the basis of his general knowledge.

For example, the content of plasticizing agent(s) and/or coalescence agent(s) can range from 0.01% to 20% by weight and in particular from 0.5% to 10% by weight, with respect to the total weight of the composition.

Gelling Agent

The nail varnish composition according to the invention can additionally comprise a gelling agent.

This gelling agent can in particular be chosen from: hydrophobic silicas, such as those described in the document EP-A-0 898 960, for example sold under the references “Aerosil R812®” by Degussa, “Cab-O-Sil TS-530 g”, “Cab-O—Sil TS-610 g” and “Cab-O-Sil TS-720®” by Cabot and “Aerosil R972®” and “Aerosil R974®” by Degussa, clays, such as montmorillonite, modified clays, such as bentones, for example stearalkonium hectorite or stearalkonium bentonite, or polysaccharide alkyl ethers (in particular for which the alkyl group comprises from 1 to 24 carbon atoms, preferably from 1 to 10, better still from 1 to 6 and more especially from 1 to 3), such as those described in the document EP-A-0 898 958.

The total proportion of gelling agent(s) in the compositions according to the invention can range from 0.01 to 15% by weight, with respect to the total weight of the composition, preferably from 0.5 to 15% by weight and better still from 0.5 to 10% by weight.

Colouring Material

The nail varnish composition according to the invention can additionally comprise one or more colouring materials chosen from soluble dyes and pulverulent colouring materials, such as pigments, pearlescent agents and glitters, well known to a person skilled in the art.

The colouring materials can be present in the composition in a content ranging from 0.01% to 50% by weight, with respect to the weight of the composition, preferably from 0.01% to 30% by weight.

Pigments should be understood as meaning white or coloured and inorganic or organic particles of any shape which are insoluble in the physiological medium and which are intended to colour the composition.

Pearlescent agents should be understood as meaning iridescent particles of any shape, in particular produced by certain molluscs in their shells or else synthesized.

The pigments can be white or coloured and inorganic or organic. Mention may be made, among inorganic pigments, of titanium dioxide, optionally surface treated, zirconium or cerium oxides, zinc, iron or chromium oxides (the iron oxides being black, yellow or red), manganese violet, ultramarine blue, chromium hydrate, ferric blue or metal powders, such as aluminium powder or copper powder.

Mention may be made, among organic pigments, of carbon black, pigments of D & C type, and lakes based on cochineal carmine of barium, strontium, calcium or aluminium.

Mention may also be made of effect pigments, such as particles comprising an organic or inorganic and natural or synthetic substrate, for example glass, acrylic resins, polyester, polyurethane, polyethylene terephthalate, ceramics or aluminas, which may or may not be covered with metal substances, such as aluminium, gold, copper or bronze, or with metal oxides, such as titanium dioxide, iron oxide or chromium oxide, or with inorganic or organic pigments, and their mixtures.

The pearlescent pigments can be chosen from white pearlescent pigments, such as mica covered with titanium oxide or with bismuth oxychloride, coloured pearlescent pigments, such as titanium oxide-coated mica covered with iron oxides, titanium oxide-coated mica covered with in particular ferric blue or with chromium oxide, or titanium oxide-coated mica covered with an organic pigment of the abovementioned type, and pearlescent pigments based on bismuth oxychloride.

Use may also be made of pigments possessing goniochromatic properties, in particular liquid crystal or multilayer pigments.

The dyes are, for example, Sudan red, DC red 17, DC green 6, β-carotene, soybean oil, Sudan brown, DC yellow 11, DC violet 2, DC orange 5 or quinoline yellow.

The colouring material can also be chosen from optical brighteners.

The composition can additionally comprise fibres optionally coated with optical brighteners.

Filler

The composition according to the invention can additionally comprise one or more fillers, in particular in a content ranging from 0.01% to 50% by weight, with respect to the total weight of the composition, preferably ranging from 0.01% to 30% by weight. Fillers should be understood as meaning colourless or white and inorganic or synthetic particles of any shape which are insoluble in the medium of the composition, whatever the temperature at which the composition is manufactured. These fillers are used in particular to modify the rheology or the texture of the composition.

The fillers can be inorganic or organic and of any shape, platelet, spherical or oblong, whatever the crystallographic form (for example sheet, cubic, hexagonal, orthorhombic, and the like). Mention may be made of talc, mica, silica, kaolin, polyamide (Nylon®) powders (Orgasol® from Atochem), poly-β-alanine powders, polyethylene powders, powders formed of tetrafluoroethylene polymers (Teflon®), lauroyl lysine, starch, boron nitride, polymeric hollow microspheres, such as those of polyvinylidene chloride/acrylonitrile, for example Expancel® (Nobel Industrie), or of acrylic acid copolymers (Polytrap® from Dow Corning), silicone resin microbeads (Tospearls® from Toshiba, for example), polyorganosiloxane elastomer particles, precipitated calcium carbonate, magnesium carbonate, basic magnesium carbonate, hydroxyapatite, hollow silica microspheres (Silica Beads® from Maprecos), glass or ceramic microcapsules, or metal soaps derived from organic carboxylic acids having from 8 to 22 carbon atoms, preferably from 12 to 18 carbon atoms, for example zinc stearate, magnesium stearate, lithium stearate, zinc laurate or magnesium myristate.

Additives

The composition can additionally comprise other ingredients commonly used in cosmetic compositions and known to a person skilled in the art as being capable of being incorporated in a nail varnish composition.

Such ingredients can be chosen from oils, waxes, agents for combating free radicals, spreading agents, wetting agents, dispersing agents, antifoaming agents, preservatives, surfactants, fragrances, neutralizing agents, stabilizing agents, antioxidants, active principles which can be chosen from the essential oils, UV screening agents/sunscreens, moisturizing agents, vitamins, proteins, ceramides, plant extracts, and the like, and their mixtures.

The compositions according to the invention can additionally comprise, as active principles, agents for caring for the nails, such as hardening agents for keratinous substances, active principles which act on the growth of the nail, such as, for example, methylsulphonylmethane, and/or active principles for treating various conditions located on the nail, such as, for example, onychomycosis.

Of course, a person skilled in the art will take care to choose this or these optional additional compounds and/or their amounts so that the advantageous properties of the composition for the use according to the invention are not, or not substantially, detrimentally affected by the envisaged addition.

According to another aspect, a subject-matter of the invention is a nail varnish product comprising: i) a container delimiting at least one compartment, the said container being closed by a closing element, and ii) a composition according to the invention which is positioned inside the said compartment.

The container can have any appropriate form. It can in particular be in the form of a bottle and can, at least in part, be made of a material such as glass. However, materials other than glass can be used, such as thermoplastics, for example PP or PE, or such as a metal.

The closing element can be coupled to the compartment by screwing into the closed position of the container. Alternatively, the coupling between the closing element and the container can be carried out other than by screwing, in particular by snapping.

The container is preferably equipped with an applicator which can be in the form of a brush composed of at least one tuft of hairs. Alternatively, the applicator is provided in a form other than a brush, for example in the form of a spatula or of a foam tip.

II—Soft Article

As specified above, the present invention relates, according to another of its aspects, to a soft article intended to be applied to the nails and/or false nails, in order to make them up and/or to care for them, comprising at least one polymeric layer, characterized in that the said polymeric layer comprises at least one drying oil, at least one film-forming polymer and at least one metal salt.

Generally, the soft article in accordance with the present invention is provided in the form of a film or laminate.

Within the meaning of the present invention, the term “soft” describes a sufficient flexibility of this film, that is to say a flexibility favourable to mechanical changes in shape of stretching type, for adjusting it at the surface of a nail.

Furthermore, it should also be understood that “soft” is understood to mean capable of changing in shape nonelastically so as to conform to the more or less rounded outline of the nail.

This ability to change shape is characterized in particular by the parameter of change in shape at break s discussed below. The article according to the invention differs in particular on this account from an article of false nail type, which is characterized by a stiffness incompatible with such a mechanical change in shape.

Another difference between the article in accordance with the invention and a false nail lies in the sensitivity of this article with regard to polar organic solvents of the acetone, ester and/or short alcohol type, such as alkyl acetates, in particular ethyl acetate.

This is because the article according to the invention can be easily removed by removal of make-up using a conventional dissolvent or a solvent, in contrast to a false nail, which is taken off. Thus, the article according to the invention can advantageously be subjected to make-up removal by organic solvents and in particular by acetone and alkyl acetates, such as ethyl acetate and their mixtures.

The invention consequently relates to an article capable of being subjected to removal of makeup using a solvent chosen from acetone, alkyl acetates, such as ethyl acetate, and their mixtures.

In all these respects, the soft article according to the invention differs, on the one hand, from conventional liquid compositions of nail varnish type due to its partially dry structure and, on the other hand, from solid products of false nail type in that the said article, before application, can be changed in shape mechanically without the necessary presence of residual solvent and can be subjected to make-up removal, in contrast to a false nail, which is affixed directly to the nail and then filed down in order to be adjusted and is subsequently removed directly from the nail in order to reencounter a nail in the normal state.

The article according to the invention can be used for making-up purposes, in which case it comprises colouring materials, or for protective purposes with regard to a varnish film. In this alternative, the polymeric layer is generally transparent.

According to one embodiment, the soft article according to the present invention exhibits a thickness ranging from 6 μm to 1 mm, in particular from 10 μm to 500 μm and more particularly still from 50 μm to 200 μm.

The targeted thickness is understood as being the thickness before application to the nail of the whole of the indissociable structure possessing one or more layers comprising in particular the polymeric layer.

In particular, if the polymeric layer is not self-adhesive and requires, on one of its faces, the presence of an adhesive layer, the targeted thickness is understood as the thickness of the combination of the polymeric layers and adhesive layers.

On the other hand, any structure fixed in detachable fashion to the soft article, in particular a thin protective film on one or other of the faces of the article, in particular a thin silicone film on the adhesive face of the article, is not taken into account in the measurement of the thickness.

A subject-matter of the invention is consequently a soft article intended to be applied to the nails and/or false nails, in order to make them up and/or care for them, as defined above, comprising at least one polymeric layer, characterized in that the said polymeric layer comprises at least one drying oil, at least one film-forming polymer and at least one metal salt and exhibits a first adhesive face comprising an adhesive material which is intended to be brought into contact with the nail and a second face opposite the first.

In particular, the soft article can additionally comprise a thin protective film in contact with the first face of the polymeric layer, to be removed prior to putting the article in place on the nail. Preferably, the face of the thin protective film in contact with the first face of the sheet is covered with a release material, in particular a silicone release material.

According to a specific embodiment, the article in accordance with the invention is coated on both its faces with an identical or different thin detachable film.

The soft article and in particular the excess can be cut to shape beforehand or cut to shape, before or after its application, according to the desired size and the desired shape with small scissors or nail clippers or by scraping the film.

The make-up product according to the invention is advantageously packaged, preferably immediately after manufacture, in a storage container, such as, for example, a soft or stiff bag, sufficiently leaktight to retain for it this not completely crosslinked aspect. It is only at the time of its use and consequently during the coming into contact thereof with air that the product gradually crosslinks in order to acquire the hardness necessary for good hold.

According to a specific form of the invention, the polymeric layer is manufactured by coating with a composition according to the invention comprising a solvent, preferably acetone, alkyl acetates, such as ethyl acetate, and their mixtures. The coating is dried with the exclusion of oxygen, preferably under nitrogen, so as to make possible the evaporation of the solvent without causing the drying oil to crosslink.

The soft article or soft article in the dry state according to the invention is provided in particular in the form of a nonliquid film which can be characterized by a high solids content. This is because the amount of dry matter in this article in the dry state, namely once applied to the nail or the false nail, is greater than or equal to 95% by weight, with respect to the total weight of the article. In other words, the amount of volatile solvent is less than or equal to 5% by weight, with respect to the total weight of the article.

The make-up product, according to another aspect touched on above, comprises an article in a partially dry form. In this case also, the make-up product is advantageously packaged in a storage container, such as, for example, a soft or stiff bag, sufficiently leaktight to retain for it this partially dry aspect. It is only at the time of its use and consequently during the coming into contact thereof with air that the product dries completely in order to acquire the dry matter content described above, in accordance with the article in accordance with the invention.

In a product according to the invention, the partially dry article advantageously has a dry matter content of greater than 80% by weight, in particular of greater than 85% by weight, and of less than 95% by weight, with respect to its total weight.

Such an article, when it is removed from the packaging of a product in accordance with the invention and exposed to ambient air, acquires a dry state as defined above at the end of 24 h.

Preferably, the amount of dry matter, or solids content, of the articles according to the invention is measured according to the method described above for the determination of the solids content of a composition of the invention.

The measurement protocol is as follows: approximately 10 g of sample of an article are deposited on a metal dish. The latter, after introducing into the dryer, is subjected to a set temperature of 120° C. for one hour. The wet weight of the sample, corresponding to the initial weight, and the dry weight of the sample, corresponding to the weight after exposure to the radiation, are measured using a precision balance.

The dry matter content is calculated in the following way:

Solids content=100×(dry weight/wet weight).

Water Uptake

The article according to the invention can advantageously be characterized in the dry state by an uptake of water, brought to 25° C., of less than or equal to 20%, in particular of less than or equal to 16% and especially of less than 10%.

According to the present patent application, “water uptake” is understood to mean the percentage of water absorbed by the article after immersing in water at 25° C. (ambient temperature) for 60 minutes. The water uptake is measured for pieces of approximately 1 cm cut out from the dry article. They are weighed (measurement of the weight W1) and then immersed in the water for 60 minutes; after immersion, the piece of film is wiped, in order to remove the excess surface water, and then weighed (measurement of the weight W2). The difference W2−W1 corresponds to the amount of water absorbed by the film.

The water uptake is equal to [(W2−W1)/W1]×100 and is expressed as percentage by weight with respect to the weight of the film.

Storage Modulus E′

Furthermore, the article according to the invention is advantageously a film having a storage modulus E′ of greater than or equal to 1 MPa, in particular ranging from 1 MPa to 5000 MPa, especially of greater than or equal to 5 MPa, in particular ranging from 5 to 1000 MPa, and more particularly of greater than or equal to 10 MPa, for example ranging from 10 to 500 MPa, at a temperature of 30° C. and a frequency of 0.1 Hz.

The storage modulus is measured by DMTA (Dynamical and Mechanical Temperature Analysis).

Viscoelasticity measurement tests are carried out with a DMTA device from Polymer TA Instruments (DMA2980 model) on a sample of article. The test specimens are cut out (for example with a hollow punch). The latter typically have a thickness of approximately 150 μm, a width of 5 to 10 mm and a working length of approximately 10 to 15 mm.

The measurements are carried out at a constant temperature of 30° C.

The sample is stressed in tension and in small strains (for example, a sinusoidal displacement of ±8 μm is imposed on it) during a frequency sweeping, the frequency ranging from 0.1 to 20 Hz. The operation is thus carried out in the linear domain, under low levels of strain.

These measurements make it possible to determine the complex modulus E*=E′+iE″ of the composition film tested, E′ being the storage modulus and E″ being the “loss” modulus.

Strain and/or Energy at Break

Advantageously, the articles according to the invention have a strain at break ∈ᵣ of greater than or equal to 5%, in particular ranging from 5 to 500%, preferably of greater than or equal to 15%, in particular ranging from 15 to 400%, and/or an energy at break per unit of volume Wb of greater than or equal to 0.2 J/cm³, in particular ranging from 0.2 to 100 J/cm³, preferably of greater than 1 J/cm³, in particular ranging from 1 to 50 J/cm³.

The strain at break and the energy at break per unit of volume are determined by tensile tests carried out on a crosslinked film with a thickness of approximately 200 μm.

To carry out these tests, the article is cut into dumbbell test specimens with a working length of 33±1 mm and a working width of 6 mm.

The cross section (S) of the test specimen is then defined as:

S=width×thickness (cm²); this cross section will be used for the calculation of the stress.

The tests are carried out, for example, on a tensile testing device sold under the name Lloyd® LR5K. The measurements are carried out at ambient temperature (20° C.).

The test specimens are drawn at a rate of displacement of 33 mm/min, corresponding to a rate of 100% elongation per minute.

A rate of displacement is thus imposed and the elongation ΔL of the test specimen and the force F necessary to bring about this elongation are measured simultaneously.

It is from these data ΔL and F that the stress a and strain c parameters are determined. A curve of stress σ=(F/S) as a function of the strain ∈=(ΔL/Lo)×100 is thus obtained, the test being carried out until the test specimen breaks, Lo being the initial length of the test specimen. The strain at break ∈ᵣ is the maximum strain of the sample before the point of break (as percentage). The energy at break per unit of volume Wb in J/cm³ is defined as the area under this stress/strain curve such that:

$$Wb = {\int }_{0}^{{ɛ}_{b}} σ , ɛ , d ɛ$$

Preferably, the soft article in accordance with the invention comprises more than 80% by weight of compounds of natural origin, preferably more than 90% by weight, preferably more than 95% by weight; preferably, the said laminate comprises exclusively compounds of natural origin.

Polymeric Layer

The drying oil(s), the film-forming polymer(s) and/or the metal salt(s) of the polymeric layer are in particular as described above in the context of the nail varnish in the liquid or gelled form.

As regards the drying oil, in the case of the soft article, the drying oil does not comprise epoxide groups. It is described as “drying oil without epoxide group” or “nonepoxidized oil”.

According to a specific embodiment, the polymeric layer of the soft article according to the invention comprises from 1 to 99% by weight, in particular from 10 to 90% by weight, especially from 30 to 70% by weight, of drying oil, with respect to the total weight of the polymeric layer.

Preferably, the polymeric layer comprises, in addition to the drying oil and the film-forming polymer, at least one metal salt for the purpose of increasing the rate of crosslinking of the drying oil,

As regards the film-forming polymer, in the case of the soft article and according to a specific embodiment, the film-forming polymer is chosen from a cellulose ether or ester. According to a particularly preferred embodiment, the film-forming polymer chosen is ethyl cellulose.

The present invention is targeted in particular at an article in accordance with the invention, characterized in that the film-forming polymer is chosen from polysaccharides or polysaccharide derivatives, preferably a cellulose alkyl ether or a cellulose ester, preferably a cellulose acetate butyrate, a cellulose acetate propionate, ethyl cellulose or ethyl guar.

According to a specific embodiment, the polymeric layer of the soft article according to the invention comprises from 1 to 99% by weight, in particular from 10 to 90% by weight, especially from 30 to 70% by weight, of film-forming polymer as dry matter, with respect to the total weight of the polymeric layer.

According to a specific embodiment, the article according to the invention comprises from 0.001 to 2% by weight, in particular from 0.01 to 1% by weight, especially from 0.1 to 0.5% by weight, of metal, with respect to the total weight of the solids content of the polymeric layer.

Still within the context of the use of the soft article, preferably, the polymeric layer in accordance with the invention comprises more than 80% by weight of, preferably more than 90% by weight of, preferably more than 95% by weight of, preferably the polymeric layer comprises exclusively, compounds of natural origin, with respect to the total weight of the polymeric layer.

Mention may be made, as optional additional compounds in the polymeric layer, of additional agents which are able to form a film. The polymeric layer can additionally comprise an additional agent which is able to form a film which can be chosen from any compound known to a person skilled in the art as being capable of fulfilling the desired purpose and can in particular be chosen from plasticizing agents and coalescence agents for the film-forming polymer.

Mention may in particular be made, alone or as mixtures, of optionally modified compounds of natural origin, such as oils comprising at least one fatty acid such as described above in the context of the alternative of the nail varnish in the liquid or gelled form.

According to a specific embodiment, the adhesive layers according to the invention comprise from 0.1 to 70% by weight, in particular from 5 to 60% by weight and more particularly from 10 to 50% by weight of plasticizer, with respect to the total weight of the solids content of the adhesive layer.

According to one embodiment of the invention, the soft article comprises an ester of caprylic and/or capric acid or an ester of caprylic and/or capric alcohol as plasticizer, the said ester being of plant origin, as defined below.

Mention may also be made of conventional plasticizers or coalescence agents, such as: glycols and their derivatives, such as diethylene glycol ethyl ether, diethylene glycol methyl ether, diethylene glycol butyl ether or diethylene glycol hexyl ether, ethylene glycol ethyl ether, ethylene glycol butyl ether or ethylene glycol hexyl ether, glycol esters, propylene glycol derivatives and in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether and diethylene glycol methyl ether, or propylene glycol butyl ether, acid esters, in particular carboxylic acid esters, such as citrates, in particular triethyl citrate, tributyl citrate, triethyl acetylcitrate, tributyl acetylcitrate or tri(2-ethylhexyl)acetylcitrate, phthalates, in particular diethyl phthalate, dibutyl phthalate, dioctyl phthalate, dipentyl phthalate or dimethoxyethyl phthalate, phosphates, in particular tricresyl phosphate, tributyl phosphate, triphenyl phosphate or tributoxyethyl phosphate, tartrates, in particular dibutyl tartrate, adipates, carbonates, sebacates, benzyl benzoate, butyl acetylricinoleate, glyceryl acetylricinoleate, butyl glycolate, camphor, glyceryl triacetate, N-ethyl-o,p-toluenesulphonamide, oxyethylenated derivatives, such as oxyethylenated oils, silicone oils, and their mixtures.

The type and the amount of plasticizing agent and/or coalescence agent can be chosen by a person skilled in the art on the basis of his general knowledge. For example, the content of plasticizing and/or coalescence agent can range from 0.01% to 20% by weight and in particular from 0.5% to 10% by weight, with respect to the total weight of the polymeric layer.

Adhesive Layer

In addition to the polymeric layer, the article according to the invention can have an adhesive outer face if the polymeric layer is not self-adhesive. Such an adhesive face is generally obtained by virtue of the presence of at least one adhesive layer, characterized in that this adhesive layer comprises at least one adhesive material.

“Material” is understood to mean, within the meaning of the present invention, a polymer or a polymer system which can comprise one or more polymers of different natures. This adhesive material can in addition comprise a plasticizer.

The latter must exhibit a certain adhesiveness defined by its viscoelastic properties.

The viscoelastic properties of a material are conventionally defined by two characteristic values which are as follows:
the elastic modulus, which represents the elastic behaviour of the material for a given frequency and which is conventionally denoted by G′,
the viscous modulus, which represents the viscous behaviour of the material for a given frequency and which is conventionally denoted by G″.

These quantities are defined in particular in the “Handbook of Pressure Sensitive Adhesive Technology”, 3^{rd} edition, D. Satas, chapter 9, pages 155 to 157.

Preferably, the adhesive materials which can be used according to the present invention exhibit viscoelastic properties which are measured at a reference temperature of 35° C. and within a certain frequency range.

In the case of adhesive materials in the form of a solution or dispersion of polymer in a volatile solvent (such as water, a short ester, a short alcohol, acetone, and the like), the viscoelastic properties of this material are measured under conditions under which it exhibits a content of volatile solvent of less than 30% and in particular a content of volatile solvent of less than 20%.

In particular, the elastic modulus of the material is measured at three different frequencies:
at low frequency, i.e. at 2×10⁻² Hz;
at an intermediate frequency, i.e. at 0.2 Hz;
at high frequency, i.e. at 2 Hz; and
   and the viscous modulus is measured at a frequency of 0.2 Hz.

These measurements make it possible to evaluate the change in the adhesiveness of the adhesive material over time.

These viscoelastic properties are measured during dynamic tests under sinusoidal stresses of low amplitude (small strains) carried out at 35° C. over a frequency range extending from 2×10⁻² to 20 Hz on a rheometer of “Haake RS50®” type under a torsional/shear stress, for example in cone-plate geometry (for example with an angle of the cone of 1°).

Advantageously, the said adhesive material meets the following conditions:
G′ (2 Hz, 35° C.)≧10³ Pa,
G′ (35° C.)≦10⁸ Pa, in particular G′(35° C.)≦ 10⁷ Pa, and
G′ (2×10⁻² Hz, 35° C.)≦3×10⁵ Pa,
   in which:
G′ (2 Hz, 35° C.) is the elastic shear modulus of the said adhesive material, measured at the frequency of 2 Hz and at the temperature of 35° C.,
G′ (35° C.) is the elastic shear modulus of the said adhesive material, measured at the temperature of 35° C., for any frequency between 2×10⁻² and 2 Hz,
G′ (2×10⁻² Hz, 35° C.) is the elastic shear modulus of the said adhesive material, measured at the frequency of 2×10⁻² Hz and at the temperature of 35° C.

In a specific form of the invention, the adhesive material also meets the following condition:
G″/G′(0.2 Hz, 35° C.)≧0.35,
   in which:
G″ (0.2 Hz, 35° C.) is the viscous shear modulus of the said adhesive material, measured at the frequency of 0.2 Hz and at the temperature of 35° C.,
G′ (0.2 Hz, 35° C.) is the elastic shear modulus of the said adhesive material, measured at the frequency of 0.2 Hz and at the temperature of 35° C.

In a specific form of the invention, the following situation prevails:
G′ (2 Hz, 35° C.)≧5×10³ Pa and in particular G′ (2 Hz, 35° C.)≧10⁴ Pa.

In another specific form of the invention, the following situation prevails:
G′ (2×10⁻² Hz, 35° C.)≦5×10⁴ Pa.

In particular, the adhesive materials according to the invention meet the following four conditions:
G′ (2 Hz, 35° C.) 10⁴ Pa,
G′ (35° C.) 10⁸ Pa, in particular G′(35° C.) δ 10⁷ Pa,
G′ (2×10⁻² Hz, 35° C.)≦5×10⁴ Pa, and
G″/G′ (0.2 Hz, 35° C.)≧0.35.

The adhesive materials can be chosen from adhesives of “Pressure Sensitive Adhesive” type, for example, such as those mentioned in the “Handbook of Pressure Sensitive Adhesive Technology”, 3^{rd} edition, D. Satas.

The adhesive materials can in particular be polymers chosen from:
polyurethanes,
acrylic polymers,
butyl rubbers, in particular from polyisobutylenes,
ethylene/vinyl acetate polymers,
polyamides, optionally modified by fatty chains,
silicone adhesives, in particular copolymers of silicone resin and of fluid silicone, such as those sold by Dow Corning under the reference Bio-PSA,
and their blends.

They can in particular be adhesive copolymers deriving from the copolymerization of vinyl monomers with polymeric entities, such as, for example, those described in U.S. Pat. No. 6,136,296. Also capable of being suitable for the invention are the adhesive copolymers described in U.S. Pat. No. 5,929,173, having a polymer backbone, with a Tg varying from 0° C. to 45° C., grafted with chains deriving from acrylic and/or methacrylic monomers and having, on the other hand, a Tg varying from 50° C. to 200° C.

These adhesive materials can also be polymers chosen from block or random copolymers comprising at least one monomer or one combination of monomers, the resulting polymer of which has a glass transition temperature below ambient temperature (25° C.), it being possible for these monomers or combinations of monomers to be chosen from butadiene, ethylene, propylene, isoprene, isobutylene and their mixtures. Examples of such materials are block polymers of styrene/butadiene/styrene, styrene/(ethylene/butylene)/styrene or styrene/isoprene/styrene type, such as those sold under the trade names “Kraton©” of Shell Chemical Co. or “Vector®” of Dexco Polymers.

The adhesive materials are, for example, chosen from polyisobutylenes exhibiting a relative molar mass Mv of greater than or equal to 10 000 and of less than or equal to 150 000. In particular, this relative molar mass is greater than or equal to 18 000 and less than or equal to 150 000.

Mention may be made, as commercial products particularly well suited to the present invention, of the polyisobutylenes with respective relative molar masses Mv of 40 000, 55 000 and 85 000 sold under the respective commercial names “Oppanol B 10®”, “Oppanol B 12®” and “Oppanol B 15®” by BASF, and their blends.

According to a specific embodiment, the adhesive material comprises at least one natural rubber, at least one tackifying resin and optionally at least one plasticizer.

Within the meaning of the invention, natural rubber is understood to mean a rubber which is obtained from plants and which may optionally have been modified, in particular by chemical reaction. Preferably, the natural rubber is not subjected to any chemical modification after extraction.

According to a favoured form of the invention, the natural rubber is obtained after removal of the aqueous phase from a natural polyisoprene latex, preferably chosen from latexes of Hevea, of Parthenium argentatum (guayule), of Solidago virgaurea minuta, of Taraxacum koksaghyz, of gutta-percha, of gutta-balata and of the sapodilla plant (Manilkara zapota), and their mixtures.

Preferably, the natural latexes are deproteinized, in particular in order to reduce the risks of allergies.

Mention may be made, as tackifying resins, of rosins or rosin derivatives, such as hydrogenated rosins, rosin esters, hydrogenated rosin esters, terpenes, aliphatic or aromatic hydrocarbon resins, phenolic resins, or styrene and coumarone/indene resins. Also regarded as tackifying resins are shellac, sandarac gum, dammar, elemi, copals, benzoin, mastic gum or their mixtures. The latter are particularly advantageous in the context of the present invention.

Plasticizer

The adhesive materials which can be employed in the layer of the present invention can additionally comprise plasticizers, preferably natural plasticizers or plasticizers of natural origin.

The plasticizers in accordance with the invention are preferably chosen from optionally modified compounds of natural origin, such as oils comprising at least one fatty acid chosen from caprylic acid, capric acid, lauric acid, myristic acid, palmitie acid, stearic acid, oleic acid, ricinoleic acid, linoleic acid, linolenic acid, arachidic acid, gadoleic acid, behenic acid, erucic acid, brassidic acid, cetoleic acid, lignoceric acid, nervonic acid and a mixture of these and in particular the oils comprising a mixture of caprylic acid and capric acid.

In particular, the oils suitable for the implementation of the invention are chosen from triglycerides composed of esters of fatty acids and of glycerol, the fatty acids of which can have varied chain lengths from C₄ to C₂₄, it being possible for the latter to be linear or branched and saturated or unsaturated. These oils are in particular heptanoic or octanoic triglycerides, groundnut, babassu, coconut, grape seed, cottonseed, maize, maize germ, mustard seed, palm, rapeseed, sesame, soybean, sunflower, wheat germ, canola, apricot, mango, castor, shea, avocado, olive, sweet almond, almond, peach, walnut, hazelnut, macadamia, jojoba, alfalfa, poppy, pumpkinseed, cucumber, blackcurrant, evening primrose, millet, barley, quinoa, rye, safflower, candlenut, passionflower, musk rose or shea butter oils or triglycerides of caprylic/capric acids, and their mixtures.

According to a specific embodiment, the adhesive layers according to the invention comprise from 0.1 to 70% by weight, in particular from 5 to 60% by weight and more particularly from 10 to 50% by weight of plasticizer, with respect to the total weight of the solids content of the adhesive layer.

According to one embodiment of the invention, the soft article comprises an ester of caprylic and/or capric acid or an ester of caprylic and/or capric alcohol as plasticizer, the said ester being of plant origin.

Caprylic and/or capric acid or alcohol is understood to mean linear or branched, preferably linear, carboxylic acids or alcohols having 8 or 10 carbons.

Within the meaning of the invention, “ester of plant origin” is understood to mean an ester which is obtained from plant starting materials or from starting materials of plant origin.

This ester can correspond to one of the following formulae (I) to (X):

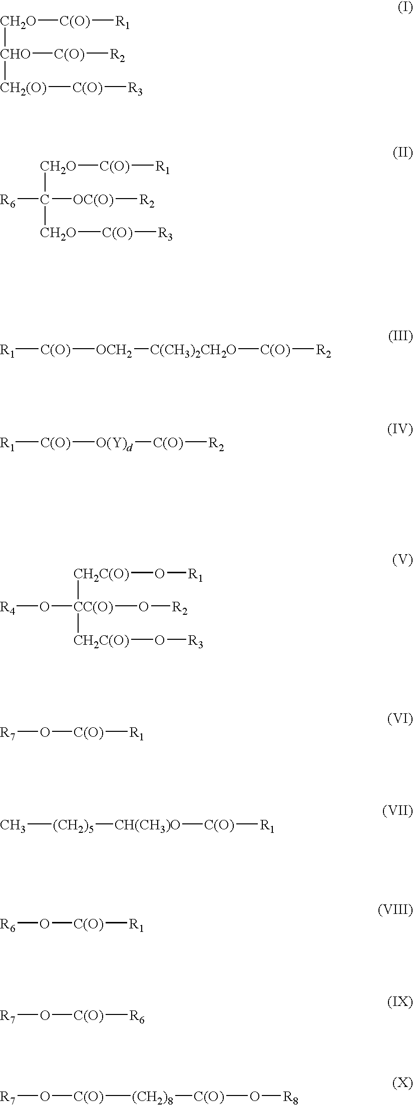

in which:

R₁, R₂ and R₃, which are identical or different, represent the hydrogen atom or a saturated and linear or branched, preferably linear, alkyl chain comprising 7 or 9 carbon atoms,

R₄ represents the hydrogen atom or an (Alk)-C(CO)— group in which (Alk) represents a linear or branched alkyl chain comprising between 1 and 5 carbon atoms,

R₆ represents a substituted or unsubstituted and linear or branched alkyl chain comprising between 1 and 18 carbon atoms, in particular between 1 and 10, or a substituted or unsubstituted and linear or branched alkenyl chain comprising between 2 and 18 carbon atoms, in particular between 2 and 10,

Y is an ethoxy, isopropoxy or propoxy group,

d is between 1 and 1000, in particular between 1 and 15, in particular equal to 1, and

R₇ and R₈, which are identical or different, represent a saturated and linear or branched, preferably linear, alkyl chain comprising 8 or 10 carbon atoms.

The ester in accordance with the invention can also comprise a mixture of different esters defined above.

Such esters are commercially available, for example from Cognis or RTD Hallstar.

According to a preferred embodiment of the invention, this plasticizer can in particular be a compound of formula (I) as defined above or, in other words, a caprylic and/or capric acid triglyceride.

According to another preferred embodiment of the invention, this plasticizer can in particular be a compound of formula (VII) as defined above or, in other words, an isooctyl caprylate and/or caprate.

According to a specific form of the invention, the adhesive layer is composed of at least one natural rubber which is optionally deproteinized, such as a natural polyisoprene latex devoid of its aqueous phase, of at least one tackifying resin among those mentioned above, preferably of at least one natural tackifying resin or tackifying resin of natural origin, and of at least one plasticizer, preferably a natural plasticizer or plasticizer of natural origin, among those mentioned above.

The adhesive layer in the article in accordance with the invention is generally in the form of a layer having a thickness of 5 to 100 μm, in particular 10 to 80 μm, preferably of 15 to 50 μm, preferably of 20 to 30 μm.

Generally, the adhesive is such that the said article cannot be removed by peeling when it is applied to the surface of a synthetic or natural nail after being in position for at least 24 hours.

Preferably, the adhesive layer in accordance with the invention comprises more than 80% by weight of, preferably more than 90% by weight of, preferably more than 95% by weight of, preferably the adhesive layer comprises exclusively, compounds of natural origin, with respect to the total weight of the adhesive layer.

Solvent of Plant Origin

The article according to the invention can comprise a solvent medium composed of one or more solvents of plant origin. In other words, according to this embodiment, the composition is then completely devoid of solvents of nonplant

Within the meaning of the invention, “solvent of plant origin” is understood to mean a solvent which is obtained directly from plants and which has not been subjected to any chemical modification after extraction.

The solvents of plant origin in accordance with the invention are preferably chosen from alcohols and/or acetates and/or lactates.

According to a preferred embodiment, the solvent of plant origin is chosen in particular from ethanol and/or ethyl acetate and/or ethyl lactate.

Preferably, it will be a mixture of ethanol and ethyl acetate.

Ethanol exclusively of plant origin is widely available from numerous suppliers, in particular from Tereos. Ethyl acetate of plant origin is available from Rhodia. Ethyl lactate is available from React-NTI under the reference Envirosolve Silver.

According to a specific embodiment, the soft article or soft article in the dry state according to the invention comprises from 1 to 20% of solvent medium by weight, in particular from 1 to 10% by weight, especially from 1 to 5% by weight, with respect to the total weight of the soft article.

Other Additives

i) Pigments and Dyes

The adhesive layer according to the invention can comprise in particular at least one organic or inorganic colouring material, in particular of pigment or pearlescent agent type, conventionally used in varnishes in the liquid or gelled form, such as described above.

The pigments can be present in a proportion of 0.01 to 20% by weight, in particular of 0.01 to 15% by weight and especially of 0.02 to 10% by weight, with respect to the total weight of the soft article.

ii) Effect Material

The adhesive layer and the polymeric layer according to the invention can comprise at least one material with a specific optical effect. This effect is different from a simple conventional colouring effect, that is to say a unified and stabilized effect such as produced by the conventional colouring materials described above, such as, for example, monochromatic pigments. Within the meaning of the invention, “stabilized” means devoid of an effect of variability in the colour with the angle of observation or else in response to a change in temperature.

This material is present in an amount sufficient to produce an optical effect perceptible to the naked eye. Advantageously, it is an effect chosen from goniochromatic, metallic and in particular mirror, soft focus, rainbow and/or thermochromic and/or photochromic effects.

For example, this material can be chosen from particles with a metallic glint, goniochromatic colouring agents, diffracting pigments, thermochromic or photochromic agents, optical brightening agents and fibres, in particular interference fibres. Of course, these various materials can be combined so as to provide the simultaneous display of two effects, indeed even of a novel effect in accordance with the invention.

Particles with a Metallic Glint

“Particles with a metallic glint” denotes particles where the nature, the size, the structure and the surface condition of the particles allow them to reflect the incident light, in particular in a noniridescent way.

The particles exhibiting a substantially flat external surface are also suitable as they can more easily give rise, if their size, their structure and their surface condition allow it, to an intense specular reflection which can then be described as mirror effect.

The particles with a metallic glint which can be used in the invention can, for example, reflect the light in all the components of the visible region without significantly absorbing one or more wavelengths. The spectral reflectance of these particles can, for example, be greater than 70% in the 400-700 nm region and better still at least 80%, indeed even 90% or even 95%.

These particles generally have a thickness of less than or equal to 1 μm, in particular of less than or equal to 0.7 μm, especially of less than or equal to 0.5 μm.

The total proportion of particles with a metallic glint is in particular of less than or equal to 20% by weight and especially of less than or equal to 10% by weight, with respect to the total weight of the first composition or of the organic and/or inorganic film.

The particles with a metallic glint which can be used in the invention are chosen in particular from:
particles of at least one metal and/or of at least one metal derivative,
particles comprising an organic or inorganic substrate, made of one or more materials, at least partially covered with at least one layer with a metallic glint comprising at least one metal and/or at least one metal derivative, and
mixtures of the said particles.

Mention may be made, among the metals which can be present in the said particles, for example, of Ag, Au, Cu, Al, Ni, Sn, Mg, Cr, Mo, Ti, Zr, Pt, Va, Rb, W, Zn, Ge, Te, Se and their mixtures or alloys. Ag, Au, Cu, Al, Zn, Ni, Mo, Cr and their mixtures or alloys (for example, bronzes and brasses) are preferred metals.

“Metal derivatives” denotes compounds derived from metals, in particular oxides, fluorides, chlorides and sulphides.

Mention may in particular be made, among the metal derivatives which can be present in the said particles, of metal oxides, such as, for example, titanium oxides, in particular TiO₂, iron oxides, in particular Fe₂O₃, tin oxides, chromium oxides, barium sulphate and the following compounds: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅ and MoS₂, and their mixtures or alloys.

According to a first alternative form, the particles with a metallic glint can be composed of at least one metal as defined above, of at least one metal derivative as defined above or also of one of their mixtures.

These particles can be at least partially covered with a layer of another material, for example a transparent material, such as in particular rosin, silica, stearates, polysiloxanes, polyester resins, epoxide resins, polyurethane resins and acrylic resins.

Mention may be made, by way of illustration of these particles, of aluminium particles, such as those sold under the names “Starbrite 1200 EAC®” by Silberline and Metalure® by Eckart.

Mention may also be made of metal powders formed from copper or from alloy mixtures, such as the references 2844 sold by Radium Bronze, metal pigments, such as aluminium or bronze, for example those sold under the names “Rotosafe 700” by Eckart, particles formed from aluminium coated with silica, sold under the name “Visionaire Bright Silver” by Eckart, and particles of metal alloy, such as powders formed from bronze (copper and zinc alloy), coated with silica, sold under the name of “Visionaire Bright Natural Gold” by Eckart.

According to a second alternative form, these particles can be particles comprising a substrate and which thus exhibit a multilayer structure, for example a two-layer structure. This substrate can be organic or inorganic, natural or synthetic, made of one or more materials, and solid or hollow. When the substrate is synthetic, it can be produced with a shape favouring the formation of a reflective surface after coating, in particular after the deposition of a layer of materials with a metallic glint. The substrate can, for example, exhibit a flat surface and the layer of materials with a metallic glint a substantially uniform thickness.

The substrate can in particular be chosen from metals and metal derivatives as mentioned above and also from glasses, ceramics, aluminas, silicas, silicates, in particular aluminosilicates and borosilicates, synthetic mica, such as fluorophlogopite, and their mixtures, this list not being limiting.

The layer with a metallic glint can entirely or partially coat the substrate and this layer can be at least partially covered with a layer of another material, for example a transparent material, in particular as mentioned above. According to a specific embodiment, this layer with a metallic glint entirely coats the substrate, directly or indirectly, that is to say with interposition of at least one metal or nonmetal intermediate layer.

The metals or metal derivatives which can be used in the reflective layer are as defined above. For example, it can be formed from at least one metal chosen from silver, aluminium, chromium, nickel, molybdenum, gold, copper, tin, magnesium and their mixtures (alloys). Use is more particularly made of silver, chromium, nickel, molybdenum and their mixtures.

Mention may more particularly be made, by way of illustration of this second type of particles, of:

Glass particles covered with a metal layer, in particular those described in documents JP-A-09188830, JP-A-10158450, JP-A-10158541, JP-A-07258460 and JP-A-05017710.

Mention may be made, by way of illustration of these particles comprising a glass substrate, of those coated respectively with silver, with gold or with titanium, in the form of platelets, sold by Nippon Sheet Glass under the names “Microglass Metashine”. Particles with a glass substrate coated with silver, in the form of platelets, are sold under the name “Microglass Metashine REFSX 2025 PS” by Toyla. Particles with a glass substrate coated with nickel/chromium/molybdenum alloy are sold under the name “Crystal Star GF 550, GF 2525” by this same company. Those coated either with brown iron oxide, on the one hand, or with titanium oxide, with tin oxide or with one of their mixtures, on the other hand, such as those sold under the name “Reflecks” by Engelhard or those sold under the reference “Metashine MC 2080GP” by Nippon Sheet Glass.

These glass particles covered with metals can be coated with silica, such as those sold under the name “Metashine series PSS1 or GPS1” by Nippon Sheet Glass.

Particles with a spherical glass substrate coated or not coated with a metal, in particular those sold under the name “Prizmalite Microsphere” by Prizmalite Industries.

The pigments of the “Metashine 1080R” range sold by Nippon Sheet Glass Co. Ltd. are also suitable for the invention. These pigments, more particularly described in Patent Application JP 2001-11340, are flakes of C-Glass glass comprising from 65 to 72% of SiO₂ which are covered with a layer of titanium oxide of rutile type (TiO₂). These glass flakes have a mean thickness of 1 micron and a mean size of 80 microns, i.e. a mean size/mean thickness ratio of 80. They exhibit blue, green, yellow or silver-coloured glints, depending on the thickness of the TiO₂ layer.

Particles comprising a borosilicate substrate coated with silver, also known as “white pearlescent agents”.

Particles with a metal substrate, such as aluminium, copper or bronze, in the form of platelets, are sold under the trade name “Starbrite” by Silberline and under the name “Visionaire” by Eckart.

Particles comprising a substrate of synthetic mica coated with titanium dioxide, for example the particles with a size of between 80 and 100 μm, comprising a substrate of synthetic mica (fluorophlogopite) coated with titanium dioxide representing 12% of the total weight of the particle, sold under the name “Prominence” by Nihon Koken.

The particles with a metallic glint can also be chosen from the particles formed of a stack of at least two layers possessing different refractive indices. These layers can be polymeric or metallic in nature and can in particular include at least one polymeric layer.

Thus, the particles with a metallic effect can be particles deriving from a multilayer polymeric film.

The choice of the materials intended to form the different layers of the multilayer structure is, of course, made so as to confer the desired metallic effect on the particles thus formed.

Such particles are described in particular in WO 99/36477, U.S. Pat. No. 6,299,979 and U.S. Pat. No. 6,387,498 and are more particularly identified below in the goniochromatic section.

Diffractive Pigments

“Diffractive pigment” denotes, within the meaning of the present invention, a pigment capable of producing a variation in colour according to the angle of observation when illuminated by white light, due to the presence of a structure which diffracts the light.

A diffractive pigment can comprise a diffraction grating capable, for example, of diffracting an incident ray of monochromatic light in defined directions.

The diffraction grating can comprise a periodic unit, in particular a line, the distance between two adjacent units being of the same order of magnitude as the wavelength of the incident light.

When the incident light is polychromatic, the diffraction grating will separate the various spectral components of the light and produce a rainbow effect.

Reference may usefully be made, relating to the structure of diffractive pigments, to the paper “Pigments Exhibiting Diffractive Effects” by Alberto Argoitia and Matt Witzman, 2002, Society of Vacuum Coaters, 45th Annual Technical Conference Proceedings 2002.

The diffractive pigment can be produced with units having different profiles, in particular triangular, symmetrical or asymmetrical, square-wave, with a constant or varying width, or sinusoidal.

The spatial frequency of the grating and the depth of the units will be chosen as a function of the degree of separation of the various orders desired. The frequency can vary, for example, between 500 and 3000 lines per mm.

Preferably, the particles of the diffractive pigment each exhibit a flattened form and in particular are in the platelet form.

The same pigment particle can comprise two intersecting, perpendicular or nonperpendicular, diffraction gratings.

A possible structure for the diffractive pigment can comprise a layer of a reflective material covered at least on one side with a layer of a dielectric material. The latter may confer better stiffness and durability on the diffractive pigment. The dielectric material can then be chosen, for example, from the following materials: MgF₂, SiO₂, Al₂O₃, AlF₃, CeF₃, LaF₃, NdF₃, SmF₂, BaF₂, CaF₂, LiF and their combinations. The reflective material can be chosen, for example, from metals and their alloys and also from nonmetallic reflective materials. Mention may be made, among the metals which can be used, of Al, Ag, Cu, Au, Pt, Sn, Ti, Pd, Ni, Co, Rd, Nb and Cr, and their compounds, combinations or alloys. Such a reflective material can, on its own, constitute the diffractive pigment, which will then be monolayer.

In an alternative form, the diffractive pigment can comprise a multilayer structure comprising a core of a dielectric material covered with a reflective layer at least on one side, indeed even a reflective layer which completely encapsulates the core. A layer of a dielectric material can also cover the reflective layer or layers. The dielectric material used is then preferably inorganic and can be chosen, for example, from metal fluorides, metal oxides, metal sulphides, metal nitrides, metal carbides and their combinations. The dielectric material can be in the crystalline, semicrystalline or amorphous state. In this configuration, the dielectric material can, for example, be chosen from the following materials: MgF₂, SiO, SiO₂, Al₂O₃, TiO₂, WO, AlN, BN, B₄C, WC, TiC, TiN, N₄Si₃, ZnS, glass particles and carbons of diamond type, and their combinations.

The diffractive pigment used can be chosen in particular from those described in United States Patent Application US 2003/0031870, published on 13 Feb. 2003.

A diffractive pigment can comprise, for example, the following structure: MgF₂/Al/MgF₂, a diffractive pigment having this structure being sold under the name “Spectraflair 1400 Pigment Silver” by Flex Products or “Spectraflair 1400 Pigment Silver FG”. The proportion by weight of the MgF₂ can be between 80 and 95% of the total weight of the pigment.

Goniochromatic Colouring Agents

Within the meaning of the invention, a goniochromatic colouring agent makes it possible to observe a change in colour, also known as “colour flop”, as a function of the angle of observation, which is greater than that which may be encountered with pearlescent agents. Use may simultaneously be made of one or more goniochromatic colouring agents.

The goniochromatic colouring agent can be chosen so as to exhibit a relatively large change in colour with the angle of observation.

The goniochromatic colouring agent can thus be chosen so that it is possible to observe, for a variation in the angle of observation of between 0° and 80° under illumination at 45°, a variation in colour ΔE of the cosmetic composition, measured in the CIE 1976 colorimetric space, of at least 2.

The goniochromatic colouring agent can also be chosen so that it is possible to observe, for illumination at 45° and a variation in the angle of observation of between 0° and 80°, a variation Dh in the angle of hue of the cosmetic composition, in the CIE 1976 plane, of at least 30°, indeed even at least 40° or at least 60°, indeed even of at least 100°.

The goniochromatic colouring agent can be chosen, for example, from multilayer interference structures and liquid crystal colouring agents.

In the case of a multilayer structure, the latter can comprise, for example, at least two layers, each layer, independently or not independently of the other layer(s), being produced, for example, from at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and their combinations.

The multilayer structure may or may not exhibit, with respect to a central layer, a symmetry with regard to the chemical nature of the stacked layers,

Examples of symmetrical multilayer interference structures which can be used in compositions produced in accordance with the invention are, for example, the following structures: Al/SiO₂/Al/SiO₂/A1, pigments having this structure being sold by DuPont de Nemours; Cr/MgF₂/Al/MgF₂/Cr, pigments having this structure being sold under the name “Chromaflair” by Flex; MoS₂/SiO₂/Al/SiO₂/MoS₂; Fe₂O₃/SiO₂/Al/SiO₂/Fe₂O₃ and Fe₂O₃/SiO₂/Fe₂O₃/SiO₂/Fe₂O₃, pigments having these structures being sold under the name “Sicopearl” by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name “Xirona” by Merck (Darmstadt). By way of examples, these pigments can be pigments with a silica/titanium oxide/tin oxide structure sold under the name “Xirona Magic” by Merck, pigments with a silica/brown iron oxide structure sold under the name “Xirona Indian Summer” by Merck and pigments with a silica/titanium oxide/mica/tin oxide structure sold under the name “Xirona Caribbean Blue” by Merck. Mention may also be made of the “Infinite Colors” pigments from Shiseido. Different effects are obtained according to the thickness and the nature of the various layers. Thus, with the structure Fe₂O₃/SiO₂/Al/SiO₂/Fe₂O₃, the colour changes from green-golden to red-grey for SiO₂ layers of 320 to 350 nm; from red to golden for SiO₂ layers of 380 to 400 nm; from purple to green for SiO₂ layers of 410 to 420 nm; and from copper to red for SiO₂ layers of 430 to 440 nm.

Use may also be made of goniochromatic colouring agents possessing a multilayer structure comprising an alternation of polymeric layers.

Mention may be made, by way of illustration of the materials from which the various layers of the multilayer structure may be formed, this list not being limiting, of: polyethylene naphthalate (PEN) and its isomers, for example 2,6-, 1,4-, 1,5-, 2,7- and 2,3-PEN, polyalkylene terephthalates, polyimides, poly(ether imide)s, atactic polystyrenes, polycarbonates, polyalkyl methacrylates and acrylates, syndiotactic polystyrene (sPS), syndiotactic poly(α-methylstyrene)s, syndiotactic polydichlorostyrene, copolymers and blends of these polystyrenes, cellulose derivatives, polyalkylene polymers, fluoropolymers, chloropolymers, polysulphones, poly(ether sulphone)s, polyacrylonitriles, polyamides, silicone resins, epoxy resins, polyvinyl acetate, poly(ether amide)s, ionomer resins, elastomers and polyurethanes. Copolymers, for example PEN copolymers (for example, copolymers of 2,6-, 1,4-, 1,5-, 2,7- and/or 2,3-naphthalenedicarboxylic acid or its esters with (a) terephthalic acid or its esters; (b) isophthalic acid or its esters; (c) phthalic acid or its esters; (d) alkane glycols; (e) cycloalkane glycols (for example, cyclohexanedimethanol diol); (f) alkanedicarboxylic acids; and/or (g) cycloalkanedicarboxylic acids), polyalkylene terephthalate copolymers and styrene copolymers, are also suitable. In addition, each individual layer can include blends of two or more of the above polymers or copolymers. The choice of the materials intended to form the various layers of the multilayer structure is, of course, made so as to confer the desired optical effect on the particles thus formed.

Mention may be made, as examples of pigments possessing a polymeric multilayer structure, of those sold by 3M under the name “Color Glitter”.

The liquid crystal colouring agents comprise, for example, silicones or cellulose ethers onto which mesomorphic groups are grafted.

Use may be made, as liquid crystal goniochromatic particles, for example, of those sold by Chemx and of that sold under the name “Helicone® HC” by Wacker.

These agents can also be in the form of disperse goniochromatic fibres. Such fibres can, for example, exhibit a size of between 50 μm and 700 μm, for example of approximately 300 μm. Use may in particular be made of interference fibres possessing a multilayer structure. Fibres possessing a multilayer structure of polymers are described in particular in the documents EP-A-0 921 217, EP-A-0 686 858 and U.S. Pat. No. 5,472,798. The multilayer structure can comprise at least two layers, each layer, independently or not independently of the other layer(s), being made of at least one synthetic polymer. The polymers present in the fibres can have a refractive index ranging from 1.30 to 1.82 and better still ranging from 1.35 to 1.75. The preferred polymers for forming the fibres are polyesters, such as polyethylene terephthalate, polyethylene naphthalate or polycarbonate, acrylic polymers, such as polymethyl methacrylate, or polyamides.

Goniochromatic fibres possessing a polyethylene terephthalate/nylon-6 two-layer structure are sold by Teijin under the name “Morphotex”.

In an alternative form, this goniochromatic colouring agent can be combined with at least one diffractive pigment.

The combination of these two materials results in a composition or a film which exhibits an increased variability in the colour and which is thus capable of allowing an observer to perceive a change in colour, indeed even a movement in colour, under numerous conditions of observation and of illumination.

The ratio by weight of the diffractive pigment with respect to the goniochromatic colouring agent is preferably between 85/15 and 15/85, better still between 80/20 and 20/80, even better still between 60/40 and 40/60, for example of the order of 50/50. Such a ratio is favourable to a strong rainbow effect and a strong goniochromatic effect being obtained.

Optical Brighteners

Optical brighteners are compounds well known to a person skilled in the art.

Such compounds are described in particular in “Fluorescent Whitening Agent, Encyclopedia of Chemical Technology, Kirk-Othmer”, vol. 11, pages 227-241, 4^{th} edition, 1994, Wiley. They can more particularly be defined as compounds which absorb essentially in the UV-A region between 300 and 390 nm and re-emit essentially between 400 and 525 nm. Mention may more particularly be made, among optical brighteners, of stilbene derivatives, in particular polystyrylstilbenes and triazinylstilbenes, coumarin derivatives, in particular hydroxycoumarins and aminocoumarins, oxazole, benzoxazole, imidazole, triazole or pyrazoline derivatives, pyrene derivatives and porphyrin derivatives, and their mixtures. Such compounds are readily available commercially.

Mention may be made, for example, of the naphthotriazole stilbene derivative sold under the trade name “Tinopal GS”, disodium 4,4′-distyrylbiphenyl sulphonate (CTFA name: disodium distyrylbiphenyl disulphonate), sold under the trade name “Tinopal CBS-X”, the cationic aminocoumarin derivative sold under the trade name “Tinopal SWN Conc.”, sodium 4,4′-bis[(4,6-dianilino-1,3,5-triazin-2-yl)amino]stilbene-2,2′-disulphonate, sold under the trade name “Tinopal SOP”, 4,4′-bis[(4-anilino-6-bis(2-hydroxyethyl)amino-1,3,5-triazin-2-yl)aminoi]stilbene-2,2′-disulphonic acid, sold under the trade name “Tinopal UNPA-GX”, 4,4′-bis[(4-anilino-6-morpholino-1,3,5-triazin-2-yl)amino]stilbene, sold under the trade name “Tinopal AMS-GX”, or disodium 4,4′-bis[(4-anilino-6-(2-hydroxyethyl)methylamino-1,3,5-triazin-2-yl)amino]stilbene-2,2′-disulphonate, sold under the trade name “Tinopal 5BM-GX”, all by Ciba Specialty Chemicals, 2,5-thiophenediylbis(5-tert-butyl-1,3-benzoxazole), sold under the trade name “Uvitex OB” by Ciba, the anionic diaminostilbene derivative as a dispersion in water sold under the trade name “Leucophor BSB Liquid” by Clariant, or the optical brightener lakes sold under the trade name “Covazur” by Wacker.

The optical brighteners which can be used in the present invention can also be provided in the form of copolymers, for example of acrylates and/or of methacrylates, grafted with optical brightener groups, as described in Application FR 9 910 942.

They can be used as is or introduced into the film in the form of particles and/or of fibres covered with the said brightener, such as those described below.

In particular, use may be made of the fibres covered with optical brightener as sold by LCW under the commercial reference Fiberlon 54 ZO₃, having a length of approximately 0.4 mm and a count of 0.5 denier.

Material with a Relief Effect

The relief effect may or may not be combined with an optical effect. A material of this type is generally present in an amount sufficient to confer a relief effect perceptible to the touch, indeed even to the naked eye. It may in particular be a rough and/or chased effect.

Material Conferring a Rough Appearance

The particles having a substantially spherical or ovoid shape can confer a soft feel on the make-up. Advantageously, the solid particles have a substantially spherical shape, in order to allow them to be satisfactorily distributed when they are applied.

The solid particles used according to the invention can have a mean size ranging from 2.5 μm to 5 mm and better still from 50 um to 2 mm. The smaller the particles, the more satisfactory the hold of the particles. The use of particles is also compatible with the production of designs.

The solid particles can be made of any material which satisfies the density properties defined above. For example, the solid particles can be made of a material chosen from glass, zirconium oxide, tungsten carbide, plastics, such as polyurethanes, polyamides, polytetrafluoroethylene or polypropylene, metals, such as steel, copper, brass or chromium, marble, onyx, jade, natural mother-of-pearl, precious stones (diamond, emerald, ruby, sapphire), amethyst or aquamarine. Use is preferably made of glass beads, such as those sold under the name “Silibeads®” by Sigmund Lindner; these beads have the additional advantage of also conferring a glossy effect and a sparkling effect on the make-up.

The solid particles, which may or may not be able to be changed in shape, can be solid or hollow, colourless or coloured and coated or uncoated.

As regards the fibres which can be used according to the invention, they can be fibres of inorganic or organic, natural or synthetic origin.

“Fibre” should be understood as meaning an object with a length L and a diameter D such that L is much greater than D, D being the diameter of the circle in which the cross section of the fibre is framed. In particular, the L/D ratio (or aspect ratio) is chosen within the range extending from 3.5 to 2500, preferably from 5 to 500 and better still from 5 to 150.

The fibres can in particular be fibres used in the manufacture of textiles and in particular silk, cotton, wool or flax fibres, fibres of cellulose, in particular extracted from wood, vegetables or algae, of rayon, of polyamide (Nylon®), of viscose, of acetate, in particular of rayon acetate, of poly(p-phenylene terephthalamide) (or of aramid), in particular of Kevlar®, of acrylic polymer, in particular of polymethyl methacrylate or of poly(2-hydroxyethyl methacrylate), of polyolefin, in particular of polyethylene or of polypropylene, of glass, of silica or of carbon, in particular in the graphite form, of polytetrafluoroethylene (such as Teflon®), of insoluble collagen, of polyesters, of polyvinyl chloride, of polyvinylidene chloride, of polyvinyl alcohol, of polyacrylonitrile, of chitosan, of polyurethane or of polyethylene phthalate, or fibres formed of a blend of polymers, such as those mentioned above, for example polyamide/polyester fibres.

Material Conferring a Chased Appearance

The inventors have also found that it is possible to package in the invention a material comprising a mixture of pyrogenic silica, metal pigment and organopolysiloxane compound, in order to confer a chased appearance thereon.

Such a mixture is described in particular in Patent Application EP 1 040 813.

iii) Material with an Olfactory Effect

Advantageously, the article according to the invention can also be endowed with olfactory properties by incorporation, in particular in at least one of the layers, of at least one fragrant material or scenting substance.

The scenting substance can be chosen from any sweet-smelling substance well known to a person skilled in the art, in particular from essential oils and/or essences.

This olfactory material can, if necessary, be introduced via a solvent-plasticizer.

“Solvent-plasticizer” is understood to mean a compound which at least partially dissolves the olfactory material and which is capable of evaporating slowly.

The solvent-plasticizer can be chosen from glycols, such as dipropylene glycol, ethyl diglycol, n-propyl glycol, n-butyl glycol, methyl diglycol or n-butyl diglycol, alcohols, such as cyclohexanol, 2-ethylbutanol, 3-methoxybutanol, 2-ethylhexanol or phenoxyethanol, esters, such as glycol monoacetate, ethyl glycol acetate, n-butyl glycol acetate, ethyl diglycol acetate, n-butyl diglycol acetate, methyl abietate, isopropyl myristate, propylene glycol diacetate or propylene glycol methyl ether acetate, or glycol ethers, such as dipropylene glycol methyl ether or dipropylene glycol butyl ether, alone or as mixtures.

Any one of the layers of the article according to the invention can also comprise one or more formulation additives commonly used in cosmetics and more especially in the nail cosmetic and/or care field. They can in particular be chosen from vitamins, trace elements, softening agents, sequestering agents, basifying or acidifying agents, wetting agents, thickening agents, dispersing agents, antifoaming agents, spreading agents, coresins, preservatives, UV screening agents, active principles, moisturizing agents, neutralizing agents, stabilizing agents, antioxidants and their mixtures.

Thus, they can in particular incorporate, as active principles, hardening or strengthening agents for keratinous substances, active principles which promote the growth of the nail, such as methylsulphonylmethane, and/or active principles for treating various conditions located on the nail, such as, for example, antimycotic or antimicrobial active principles.

The amounts of these various ingredients are those conventionally used in this field, for example from 0.01 to 20% by weight, in particular from 0.01 to 10% by weight, with respect to the total weight of the soft sheet and/or adhesive material.

The invention is illustrated in more detail in the following examples, which are presented by way of illustration and without implied limitation of the invention.

Unless otherwise indicated, the amounts are given as percentages by weight of starting material, with respect to the total weight of the composition.

### EXAMPLES

### Example 1

| | |
|---|---|
| Ethyl guar | 10% |
| Blown linseed oil “Lin Soufflé | 10% |
| CV 5P”⁽¹⁾ | |
| Manganese octoate⁽²⁾ | 0.6% |
| Zirconium octoate⁽³⁾ | 1.8% |
| Calcium octoate, neutral⁽⁴⁾ | 1.6% |
| Ethyl acetate | 76% |

⁽¹⁾sold by Novance
⁽²⁾Octa-Soligen Manganese 6 HS, sold by OMG Borchers GmbH
⁽³⁾Octa-Soligen Zirconium 12 HS, sold by OMG Borchers GmbH
⁽⁴⁾Octa-Soligen Calcium 5 HS, sold by OMG Borchers GmbH

### Reference Example

### Without Metal Salt

| | |
|---|---|
| Ethyl guar | 10% |
| Ethyl acetate | 90% |

Procedure:

The ethyl guar and the linseed oil are dissolved in the ethyl acetate.

The metal salts are subsequently added.

Films of these two compositions are coated to a wet thickness of 50 μm on a contrast chart.

Result:

After drying at 30° C. for 24 hours, the composition of Example 1 forms a solid and glossy film which adheres to the contrast chart.

The reference composition forms a less adherent film which is successfully unstuck from the contrast chart.

### Example 2

In this example, a first composition (see following table) is coated to a wet thickness of 300 μm on a release liner (“Scotchpak 1022 Release Liner 3.0 mil”, available from 3M).

| | |
|---|---|
| Ethyl cellulose⁽¹⁾ | 10% |
| Linseed oil⁽²⁾ | 10% |
| Ethyl acetate of plant origin⁽³⁾ | 75% |
| Manganese octoate⁽⁴⁾ | 0.6% |
| Zirconium octoate⁽⁵⁾ | 1.8% |
| Calcium octoate⁽⁶⁾ | 1.6% |
| Pigments | 1.0% |

⁽¹⁾Ethocel Standard 45 Premium, sold by Dow Chemical.
⁽²⁾Lin Technipol Supra, sold by Novance.
⁽³⁾sold by Rhodia.
⁽⁴⁾Octa-Soligen Manganese 6 HS, sold by Borchers.
⁽⁵⁾Octa-Soligen Zirconium 12 HS, sold by Borchers.
⁽⁶⁾Octa-Soligen Calcium 5 HS, neutral, sold by Borchers.

The film of the first composition is dried under a stream of nitrogen so as to make possible the evaporation of the solvent without causing the linseed oil to crosslink, The film of the first composition is subsequently brought into contact with an adhesive layer of PSA type (Bio-PSA 7-4602, Dow Corning) coated beforehand onto a release liner. A pressure is exerted in order to cause the films to adhere to one another. The laminate obtained is cut to shape in order to have the shape of nails and is then introduced into a substantially leaktight packaging in order to prevent the crosslinking of the drying oil from continuing. A product for making up and/or caring for the nails and/or false nails is then obtained in which the partially dry soft article is packaged.

During application to the nails, the packaging is opened immediately before applying the laminate to the nails. The release liners are carefully removed. Before crosslinking of the linseed oil, the linseed oil allows the laminate to be easily stretched during application to the nails. The polymeric layer gradually becomes harder over time.

1. Nail varnish, comprising at least one drying oil, at least one film-forming polymer and at least one metal salt, and comprising less than 5% by weight of nitrocellulose dry matter, with respect to the total weight of the nail varnish.

2. Nail varnish, comprising at least one drying oil, at least one film-forming polymer and at least one metal salt, said film-forming polymer being chosen from polysaccharide ethers and esters.

3. Nail varnish according to claim 1, wherein the drying oil is chosen from linseed oil, China wood oil, oiticica oil, vernonia oil, poppy seed oil, pomegranate seed oil or calendula oil; the esters of these vegetable oils; the alkyd resins obtained from these vegetable oils; and their mixtures; it being possible for the said drying oil optionally to be modified by chemical reaction.

4. Nail varnish according to claim 1, wherein the drying oil is refined or blown linseed oil.

5. Nail varnish according to claim 1, wherein the drying oil is present in a content ranging from 5 to 95% by weight, with respect to the total weight of the nail varnish.

6. Nail varnish according to claim 1, wherein the metal salt comprises at least one metal salt chosen from manganese, calcium, zirconium, zinc, strontium, lithium, cerium and vanadium salts.

7. Nail varnish according to claim 6, wherein the metal salt is an organic salt exhibiting the following formula (I): 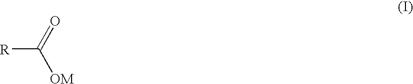
in which:
M is chosen from manganese, calcium, zirconium, zinc, strontium, lithium, cerium and vanadium; and
R is a linear or branched (C₁-C₂₅)alkyl radical which can optionally comprise from 1 to 3 unsaturations.

8. Nail varnish according to claim 1, said nail varnish comprising at least one manganese salt.

9. Nail varnish according to claim 1, wherein the metal salt comprises at least two different metal salts chosen from manganese, calcium, zirconium, zinc, strontium, lithium, cerium and vanadium salts.

10. Nail varnish according to claim 1, wherein the metal salt comprises at least one manganese salt and at least one zirconium salt.

11. Nail varnish according to claim 1, wherein the metal salt comprises at least one manganese salt, at least one zirconium salt and at least one calcium salt.

12. Nail varnish according to claim 1, wherein the metal salt(s) is (are) present in a content ranging from 0.001 to 2% by weight, with respect to the solids content.

13. Nail varnish according to claim 1, wherein the film-forming polymer is chosen from polysaccharides and polysaccharide derivatives.

14. Nail varnish according to claim 1, wherein the film-forming polymer is chosen from ethyl guar and ethyl cellulose.

15. Nail varnish according to claim 1, wherein the total content of film-forming polymer is between 1 and 60% by weight, with respect to the total weight of the nail varnish.

16. Nail varnish according to claim 2, wherein the drying oil is chosen from linseed oil, China wood oil, oiticica oil, vernonia oil, poppy seed oil, pomegranate seed oil or calendula oil; the esters of these vegetable oils; the alkyd resins obtained from these vegetable oils; and their mixtures.

17. Nail varnish according to claim 2, wherein the drying oil is refined or blown linseed oil.

18. Nail varnish according to claim 2, wherein the drying oil is present in a content ranging from 5 to 95% by weight, with respect to the total weight of the nail varnish.

19. Nail varnish according to claim 2, wherein the metal salt comprises at least one metal salt chosen from manganese, calcium, zirconium, zinc, strontium, lithium, cerium and vanadium salts.

20. Nail varnish according to claim 19, wherein the metal salt is an organic salt exhibiting the following formula (I): 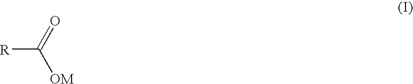
in which:
M is chosen from manganese, calcium, zirconium, zinc, strontium, lithium, cerium and vanadium; and
R is a linear or branched (C₁-C₂₅)alkyl radical.

21. Nail varnish according to claim 2, said nail varnish comprising at least one manganese salt.

22. Nail varnish according to claim 2, wherein the metal salt comprises at least two different metal salts chosen from manganese, calcium, zirconium, zinc, strontium, lithium, cerium and vanadium salts.

23. Nail varnish according to claim 2, wherein the metal salt comprises at least one manganese salt and at least one zirconium salt.

24. Nail varnish according to claim 2, wherein the metal salt comprises at least one manganese salt, at least one zirconium salt and at least one calcium salt.

25. Nail varnish according to claim 2, wherein the metal salt(s) is (are) present in a content ranging from 0.001 to 2% by weight, with respect to the solids content.

26. Nail varnish according to claim 2, wherein the film-forming polymer is chosen from cellulose acetate butyrates, cellulose acetate propionates, cellulose acetate, ethyl celluloses, ethyl guars and their blends.

27. Nail varnish according to claim 2, wherein the film-forming polymer is chosen from ethyl guar and ethyl cellulose.

28. Nail varnish according to claim 2, wherein the total content of film-forming polymer is between 1 and 60% by weight, with respect to the total weight of the nail varnish.

29. Nail varnish according to claim 2, said nail varnish comprising less than 5% by weight of nitrocellulose dry matter, with respect to the total weight of the nail varnish.

30. Cosmetic method for making up and/or for the nontherapeutic care of the nails comprising the application, to the nails, of at least one layer of nail varnish comprising at least one drying oil, at least one film-forming polymer and at least one metal salt, and comprising less than 5% by weight of nitrocellulose dry matter, with respect to the total weight of the nail varnish.

31. Cosmetic method for making up and/or for the nontherapeutic care of the nails comprising the application, to the nails, of at least one layer of nail varnish comprising at least one drying oil, at least one film-forming polymer and at least one metal salt, said film-forming polymer being chosen from polysaccharide ethers and esters.

32. Soft article intended to be applied to the nails and/or false nails, in order to make them up and/or to care for them, comprising at least one polymeric layer, wherein the said polymeric layer comprises at least one drying oil, at least one film-forming polymer and at least one metal salt.

33. Article according to claim 32, wherein the drying oil is chosen from linseed oil, China wood oil, oiticica oil, vernonia oil, poppy seed oil, pomegranate seed oil or calendula oil; the esters of these vegetable oils; the alkyd resins obtained from these vegetable oils; and their mixtures.

34. Article according to claim 32 wherein the drying oil is a nonepoxidized drying oil.

35. Article according to claim 32, wherein the polymeric layer comprises from 1 to 99% by weightof drying oil, with respect to the total weight of the polymeric layer.

36. Article according to claim 32, wherein the film-forming polymer is chosen from polysaccharides or polysaccharide derivatives.

37. Article according to claim 32, wherein the polymeric layer comprises from 1 to 99% by weight, with respect to the total weight of the polymeric layer.

38. Article according to any one of claim 32, wherein the metal salt comprises at least one metal salt chosen from manganese, calcium, zirconium, zinc, strontium, lithium, cerium and vanadium salts.

39. Article according to claim 32, wherein the polymeric layer comprises from 0.001 to 2% by weight, with respect to the solids content of the polymeric layer.

40. Article according to claim 32, wherein the said article additionally comprises at least one adhesive layer comprising at least one natural rubber, and at least one natural tackifying resin.

41. Article according to claim 32, wherein the soft article exhibits a thickness ranging from 6 μm to 1 mm.

42. Article according to claim 32, wherein the article comprises more than 80% by weight of compounds of natural origin, with respect to the total weight of the said article.

43. Article according to claim 32, wherein the article is capable of being subjected to removal of make-up using a solvent chosen from acetone, alkyl acetates, and their mixtures.

44. Article according to claim 32, wherein the weight of the solids content of the said article is greater than or equal to 95% by weight, with respect to the total weight of the article.

45. Product for making up and/or caring for the nails and/or false nails comprising, in a substantially airtight packaging, at least one soft article comprising at least one polymeric layer, wherein the said polymeric layer comprises at least one drying oil, at least one film-forming polymer and at least one metal salt, the packaging being such that the article is found therein stored in a partially dry form.

46. Method for making up and/or for caring for the nails and/or false nails comprising at least one stage which consists in applying, to a natural nail and/or false nails, a dry article comprising at least one polymeric layer, wherein the said polymeric layer comprises at least one drying oil, at least one film-forming polymer and at least one metal salt, or an article in a partially dry form included in a substantially airtight packaging of a product, said article comprising at least one polymeric layer, wherein the said polymeric layer comprises at least one drying oil, at least one film-forming polymer and at least one metal salt.
